(19) United States
(12) Patent Application Publication
(10) Pub. No.: US 2022/0351846 A1
(43) Pub. Date: Nov. 3, 2022

(54) SYSTEM AND METHOD FOR DETERMINING RETENTION OF CAREGIVERS

(71) Applicant: MatrixCare, Inc., Bloomington, MN (US)

(72) Inventors: Krishna BALA, Bloomington, MN (US); Michael F. DOLCE, Bloomington, MN (US); Navin GUPTA, Bloomington, MN (US); Vivek KUMAR, Bloomington, MN (US); Iosif Costa PETROU, Bloomington, MN (US); Roman PYSMENNYY, Bloomington, MN (US); Julie Anne WOLFF, Bloomington, MN (US); Samsudhin H, Kancheepuram (IN)

(21) Appl. No.: 17/733,285

(22) Filed: Apr. 29, 2022

Related U.S. Application Data
(60) Provisional application No. 63/182,709, filed on Apr. 30, 2021.

(51) Int. Cl. G16H 40/20; G06Q 10/10; G06N 20/00

(52) U.S. Cl. CPC G16H 40/20; G06Q 10/1053; G06N 20/00

(57) ABSTRACT

A system and method to determine a retention prediction for a caregiver is disclosed. The system includes a database of caregiver data and patient data. The set of caregiver data and patient data are normalized to create a modified set of caregiver and patient data. The modified set of caregiver and patient data defines a set of parameters or inputs from the set of caregiver and patient data and a corresponding employment status. An analysis is performed of parameters correlated with an employment status for each of the caregivers. Based on the correlation and the modified set of caregiver and patient data, a training set of caregiver data is generated that includes at least one parameter that correlates with employment status. The machine learning model is trained using the training set. The training allows a prediction of an employment status associated with the parameter. The accuracy of the trained machine learning model is evaluated.

### PRIORITY CLAIM

The present disclosure claim priority to and the benefit of U.S. Provisional Ser. No. 63/182,709 filed on Apr. 30, 2021. The contents of that application are hereby incorporated in their entirety.

### TECHNICAL FIELD

The present disclosure relates generally to methods of improving retention of health care professionals, and more specifically to a system and method for evaluating and recommending actions for the retention of caregivers in a home care and home health environment.

### BACKGROUND

Caregivers may be defined as persons tasked with providing in home and clinical healthcare to patients. The caregiver profession has numerous challenges. Thus, in out-of-hospital healthcare settings, including, for example, home care, home health, and hospice settings, caregiver turnover can be above 80%. This high turnover results in costly efforts by care providers related to separation, loss of productivity, recruitment, training, and onboarding of caregivers.

Existing human resource systems track attrition and turnover rates, but cannot determine, in real-time, the likelihood that any given caregiver will depart because of the complexity of the scenarios that lead to caregiver departure. Similarly, because of the innumerable factors that lead to caregiver departure, administrators and recruiters cannot determine which factors to consider in determining the likelihood that a particular caregiver will depart.

In the caregiver environment, a client may be defined as a patient in an out-of-hospital care setting. An agency is either: 1) a home health agency; 2) a private duty agency; or 3) a hospice. A private duty agency or a home care agency is a non-clinical caregiver who visits home and provides assistance with day to day tasks. Home health care is a field that focuses on the skilled medical aspects of care (skilled nursing, PT/OT, social work). A hospice is a facility with skilled medical care to help clients with terminal illnesses.

The number of various care settings makes predicting retention of caregivers as a general rule difficult. In contrast, the jobs of staff in skilled nursing facilities face a different set of challenges where staff are hired to serve residents in the facilities. For example, there are different issues for different care givers. For example, there may be differences in different care settings. There may be variety in the number of home visits required by caregivers. Type of service provided (e.g., private duty services in senior living facilities or skilled nursing in hospice). Further, traditional factors such as how pay is structured (salary, hourly, per patient) may influence retention. The home care industry seems to have higher turnover due to an ability of caregivers to seek lower-acuity jobs in retail and other competing businesses. Home care caregivers tend to be hourly wage earners that may not get full time engagements and might find similar hourly rates with potentially less challenging jobs in industries such as retailing. Given the wide variety of factors, turnover is difficult to predict and agencies do not have assurances during hiring process of matching caregiver roles with candidates that are likely to stay on the job. Similarly, home health caregivers might also be challenged with heavier case loads that comprise higher acuity patients with comorbidities or multiple health conditions.

There is therefore a need for a system that provides predictions as to retention of caregivers. There is a further need for a system that can evaluate input factors relating to a caregiver to determine their influence on retention of caregivers. There is also a need for a system that uses retention scores for caregivers to assist in management of caregivers. Finally, there is a need for prescribing potential action to take in order to increase the retention of a caregiver in such environments.

### SUMMARY

One disclosed example is a computer-implemented method for training a machine learning model to predict caregiver retention. A set of data is received from at least one database storing caregiver data. The set of caregiver data is normalized to create a modified set of caregiver data. The modified set of caregiver data defines at least one parameter for each of the plurality of caregivers from the set of caregiver data and a corresponding employment status for each of the plurality of caregivers. Based on the correlation and the modified set of caregiver data, a training set of caregiver data is generated that includes at least one parameter that correlates with employment status. The training data is split into a first set of data to train and validate a machine learning model and a second set of data to test the trained machine learning model. The machine learning model is trained using the first set of data. The training includes predicting an employment status associated with the at least one parameter. The accuracy of the trained machine learning model is evaluated with the second set of data.

Another disclosed example is a method of generating a retention score for a caregiver. A machine learning model is trained using a first input factor to predict retention. The training includes providing a dataset of caregivers having the first input factor and an associated employment status. The machine learning model is evaluated to determine the associated employment status generated by the model meets a predetermined accuracy level. An input factor relating to the caregiver is input to the trained machine learning model to predict retention. A retention score of the caregiver is determined based on the predicted retention.

Another disclosed example is a method for determining retention scores for a plurality of caregivers. Caregiver data is collected from a caregiver database, the caregiver data including an input factor relating to employment status for each of the plurality of caregivers. The input factors are input into a machine learning model trained using the input factor to predict retention. The training includes providing a dataset of caregivers having the input factor and an associated employment status. A retention score of each of the plurality of caregivers is generated from the machine learning model. The retention scores of at least some of the plurality of caregivers is displayed on a display.

Another disclosed example is a system for training a machine learning model to predict retention of a caregiver. The system includes a data communication interface receiving a set of data from at least one database storing caregiver data. A data correlation module normalizes the set of caregiver data to create a modified set of caregiver data. The modified set of caregiver data defines at least one parameter for each of the plurality of caregivers from the set of caregiver data and a corresponding employment status for each of the plurality of caregivers. A machine learning module generates, based on the correlation and the modified set of caregiver data, a training set of caregiver data that includes at least one parameter that correlates with employment status. The machine learning module trains the machine learning model using a first set of data split from the training set. The training includes predicting an employment status associated with the at least one parameter. The machine learning module evaluates the accuracy of the trained machine learning model from a second set of data split from the training set.

Another disclosed example is a system for generating a retention score for a caregiver. The system includes an interface for collecting caregiver data from a caregiver database. The caregiver data includes an input factor relating to an employment status of the caregiver. A machine learning model is trained using the input factor to predict retention. The training includes providing a dataset of caregivers having the input factor and an associated employment status. The machine learning model determines a retention score of the caregiver. A display displays the retention score of the caregiver.

Another disclosed example is a system for determining retention scores for a plurality of caregivers. The system includes an interface for collecting caregiver data from a caregiver database. The caregiver data includes an input factor relating to employment status for each of the plurality of caregivers. The system includes a machine learning model trained using the input factor to predict retention. The training includes providing a dataset of caregivers having the input factor and an associated employment status. The machine learning model determines a retention score of each of the plurality of caregivers. A display displays the retention scores of at least some of the plurality of caregivers.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1 shows a data collection system for determining caregiver satisfaction, predicting retention and recommending actions to take to improve retention;

FIG. 2 is a high-level block diagram illustrating an example of a computing device used in either as a client device, application server, and/or database server;

FIG. 3 is a flow diagram of an example machine learning training process to determine retention prediction based on two example factors;

FIG. 4 is a block diagram of the input sources for use of a trained machine learning module to determine a prediction of retention for a caregiver

FIG. 5 is a block diagram of a routine using retention prediction for scheduling caregivers;

FIG. 6 is a block diagram of a routine using retention prediction for evaluating potential candidates for a caregiver position;

FIG. 7A is a screen image of an example interface for management of an agency where an agency (corporate center) may have multiple offices;

FIG. 7B is a screen image of the example interface in FIG. 7A with various pulldown menus shown;

FIG. 7C is a screen image of an example management interface for a specific office of an example agency;

FIG. 7D is screen image of an example window accessed from the example management interface in FIG. 7C; and

FIGS. 8A-8D are screen images of an example scheduler interface that incorporates the retention prediction into scheduling.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to a system that provides analysis of the risk of caregiver retention and provides guidance to users (e.g. human resource managers and managers in general) on the potential root cause behind a change in retention. The system allows a determination if a caregiver is highly retainable or at high-risk to leave. The system provides analysis that outputs key factors for retention. This allows visibility into key factors underlying caregiver turnover and attrition. The system provides a dashboard that allows an agency to monitor caregiver performance, be alert to developing trends related to caregiver turnover and caregiver support, and provides alerts to deviations in behavior norms. The retention analysis may be employed to provide positive reinforcement and other interventions for struggling caregivers. The analysis also provides insight into when a caregiver is likely to depart.

In relation to hiring practices, the analysis system determines key data metrics that will help determine a potential caregiver's likelihood to remain on the job. The analysis system provides metrics to predict caregiver retention before hiring and while employed. The metrics may also distinguish between temporary decrease in job satisfaction and long-term issue that would lead to caregiver turnover.

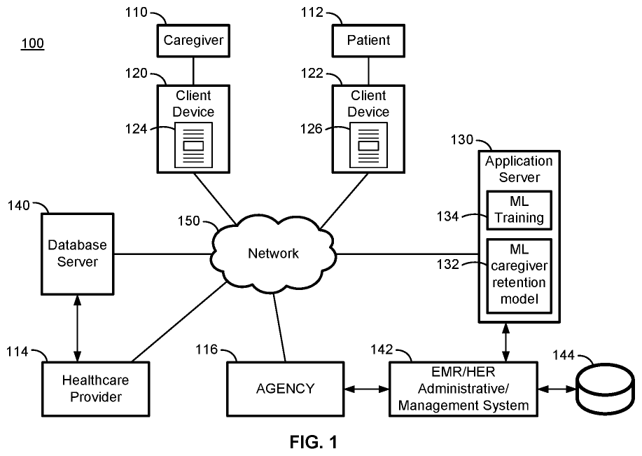

FIG. 1 shows a data collection system 100 relating to a caregiver environment. The system 100 includes different actors including caregivers such as a caregiver 110, and patients such as a patient 112, health care organizations 114, and caregiver employers such as an agency 116. Each of the actors 110, 112, 114, and 116 may provide data that relates to the analysis of retention of the caregivers 110.

The caregiver 110 is responsible for the care of a patient or patients such as the patient 112. The caregiver 110 will operate a computing device 120 that executes an application 124 allowing the caregiver 110 to record actions performed in the caregiving role. The application 124 may also include interfaces that provide information relating to the patient for the caregiver. The application 124 may collect data from interactions of the caregiver 110 with the computing device 120 or provide interfaces to collect data from the caregiver 110. The data collected from the application 124 may also include metadata such as time stamps, location, environmental factors and the like that are recorded by the agency 116 as part of the employment data relating to the care giver. The data collected from the application 124 may also include clinical information related to the health of the patient. The caregiver computing device 120 is typically configured to wirelessly communicate with the agency 116 via network 150.

The patient 112 can also have access to a computing device 122 that includes an application 126. The application 126 may be provided by the healthcare provider 114 to assist in maintaining the health of the patient. For example, the application 126 may assist in reminding the patient 112 of types of medications and the times to take such medications. The application 126 may be interfaced with sensors monitoring patient physiology or sensors on health treatment devices such as respiratory therapy devices, inhalers, or portable oxygen concentrators to provide additional data relating to the health of the patient. The application 126 may also generate interfaces for the patient 112 to provide data relating to the caregiver 110.

The caregiver 110 can also capture clinical information about the patient, including, but not limited to, vital signs (e.g., blood pressure, temperature, pulse oximetry), medication administration, progress notes in the form of voice transcriptions or typed text, structured and unstructured care plans that prescribe a plan of care for the resident usually in the form of problems, interventions and goals. This clinical data will reside in an agency management system 142 also sometimes called the Electronic Healthcare Medical Record (EMR) or Electronic Healthcare Record (EHR) system. The agency management system 142 contains a management and administration console, application or module that manages caregiver assignments and schedules and typically also stores clinical patient information including that which is entered into application 124. A dedicated agency database 144 stores relevant data for the agency management system 142. The data present in the agency management system 142 also comprises clinical data that is brought over from the healthcare provider system 114. This agency management database 144 can also contain financial information related to the patient and the agency. Examples of patient centric financial information include as examples: insurance payor information, claims submitted and cleared, receivables, payables, private payor information, credit card or bank account details.

In this example, a caregiver retention analysis server 130 provides analysis based on a machine learning retention model 132. The machine learning model 132 is trained via a machine learning training module 134. The training module 134 collects training data from the different actors that is the used to train the machine learning retention module. The data may be obtained via the network 150 from different sources. As will be explained, the machine learning retention model 132 determines a retention prediction score for the retention of caregivers such as the caregiver 110.

The health care organization 114 refers generally to any organization that may provide health care such as an application services provider or an organization that may be part of the agency 116, or closely aligned with the agency 116. The healthcare organization 114 may include institutions such as a hospital, a physician organization, or skilled nursing facility and the like. The health care organization 114 has access to a patient database 140 that includes medical records of patients of the health care organization such as the patient 112. Patient clinical and financial information are stored in the EHR and administrative system 142. The agency 116 has access to an administrative management and EHR system 142 that accesses the employee and patient information (clinical and financial) database 144. This database can be a composite or collection of many individual databases. The administrative management system 142 supports the needs of the caregiving organization such as scheduling, human resources, and the like. Patient information for patients managed by the agency is typically stored by the agency administrative management and EHR system 142. However, such patient data may also be separately stored in some cases. A wholistic view of the patient's health condition can be kept in the agency database 144 using locally available information already present and also querying or integrating with information from other databases like 140 that is associated with the healthcare provider 114. In this example, the agency 116 employs or supervises caregivers 110. In this example, patients such as the patient 112 may contract with the agency 116 for services of the caregivers 110. Thus, the patients 112 are clients of the agency 116. In this example, the administrative management system 142 may communicate with the application server 130 to determine retention prediction scores to assist in management and supervision of caregivers.

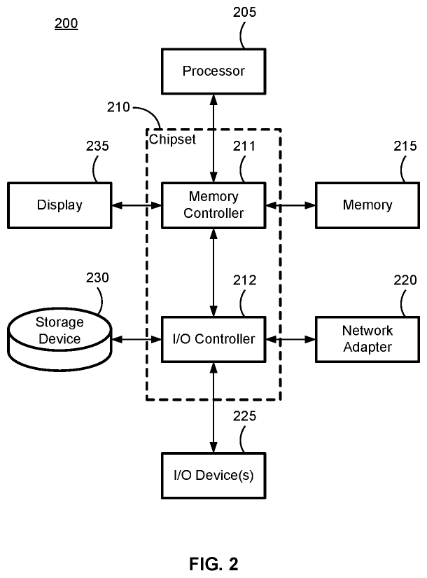

The computing devices 120 and 122 are computer systems. An example physical implementation of the computing devices 120 and 122 is described more completely below with respect to FIG. 2. The computing devices 120 and 122 are configured to wirelessly communicate with the server 130 via the network 150. With network access, the computing devices 120 and 122 transmit to the system 100 the user's geographical location and the time of different events, as well as information describing different event such as receiving care, taking medication, receiving automated treatment and the like.

Regarding user location and event times, the computing devices 120 and 122 may determine the geographical location and time of events through use of information about the cellular or wireless network 150 to which it is connected. For example, is standard electronic visit verification processes used to capture electronic visit verification data. Alternatively, the current geographical location of the computing devices 120 and 122 may be determined by directly querying the software stack providing the network 150 connection. A Global Positioning System (GPS) receiver may also be used for this purpose that is embedded in the client device 120. Alternatively, the geographical location information may be obtained by pinging an external web service (not shown in FIG. 1) made accessible via network 150.

In addition to communicating with the application server 130, computing devices 120 and 122 connected wirelessly to the retention analytics system 100 may also exchange information with other connected computing devices 120 and 122. For example, through a client software application 126, a healthcare provider 114 or an application running at the healthcare provider 114 may receive notifications describing a recent event about the patient 112, then in response send a recommendation to the patient 112 for treatment. Similarly, through application 126, patients may communicate with their health care providers 114 and other patients.

The applications 124 and 126 may provide a user interface that is displayed on a screen of the computing devices 120 and 122 and allows a user to input commands to control the operation of the application. The applications 124 and 126 may be coded as a web page, series of web pages, or content otherwise coded to render within an internet browser. The applications 124 and 126 may also be coded as a proprietary application configured to operate on the native operating system of the computing devices 120 and 122.

The computing devices 120 and 122 may communicate with local devices such as treatment devices using a network adapter and either a wired or wireless communication protocol, an example of which is the Bluetooth Low Energy (BTLE) protocol. BTLE is a short-ranged, low-powered, protocol standard that transmits data wirelessly over radio links in short range wireless networks. After another device and the computing device have been paired with each other using a BTLE passkey, the device automatically synchronizes and communicates information to the computing device. In other implementations, other types of wireless connections are used (e.g., infrared or IEEE 802.11).

The application server 130 is a computer or network of computers. Although a simplified example is illustrated in FIG. 1, typically the application server will be a server class system that uses powerful processors, large memory, and faster network components compared to a typical computing system used, for example, as the computing devices 120 and 122. The server typically has large secondary storage, for example, using a RAID (redundant array of independent disks) array and/or by establishing a relationship with an independent content delivery network (CDN) contracted to store, exchange and transmit data. Additionally, the computing system includes an operating system, for example, a UNIX operating system, LINUX operating system, or a WINDOWS operating system. The operating system manages the hardware and software resources of the application server 130 and also provides various services, for example, process management, input/output of data, management of peripheral devices, and so on. The operating system provides various functions for managing files stored on a device, for example, creating a new file, moving or copying files, transferring files to a remote system, and so on.

The application server 130 includes a software architecture for supporting access and use of the analytics system 100 by many different client devices 120 and 122 as well as workstations that are part of systems such as the administrative system 142 through the network 150, and thus at a high level can be generally characterized as a cloud-based system. Access to the administrative system 142 may also be made via a mobile phone or tablet with appropriate connection and security software. The application server 130 generally provides a platform to report relevant input data to the application server 130.

Generally, the application server 130 is designed to handle a wide variety of data. The application server 130 includes logical routines that perform a variety of functions including checking the validity of the incoming data, parsing and formatting the data if necessary, passing the processed data to a database server for storage, and confirming that the database servers 140 or 144 have been updated.

The database servers 140 and 144 store and manages data at least in part on a patient by patient basis. Towards this end, the database servers 140 and 144 create a patient profile for each user. The patient profile is a set of data that characterizes a patient such as the patient 112. The patient profile may include identity information about the patient such as age, gender, current rescue medication, current controller medication, notification preferences, a controller medication adherence plan, a relevant medical history, and a list of non-patient users authorized to access to the patient profile. The agency 116 will primarily rely on patient data stored in the database 144 where the data in the database 144 may have been created by caregiver 110 using the device 120 or collected or integrated from database 140 for the healthcare provider 114.

The application server 130 may create profiles for health care providers such as the health care provider 114. A health care provider profile may include identifying information about the health care provider, such as the office location, qualifications and certifications, and so on. The health care provider profile also includes information about their patient population. The provider profile may include access to all of the profiles of that provider's patients, as well as derived data from those profiles such as aggregate demographic information, rescue and controller medication event patterns, and so on. This data may be further subdivided according to any type of data stored in the patient profiles, such as by geographic area (e.g., neighborhood, city) over by time period (e.g., weekly, monthly, or yearly). Data from such profiles including relevant information from the database 144 may be used for input factors for determining caregiver retention.

The database servers 140 and 144 store patient and provider data related data such as profiles, medication events, patient medical history (e.g., electronic medical records). Patient and provider data are encrypted for security and is at least password protected and otherwise secured to meet all Health Insurance Portability and Accountability Act (HIPAA) requirements. Any analyses (e.g., asthma risk analyses) that incorporate data from multiple patients (e.g., aggregate rescue medication event data) and are provided to users can be de-identified so that personally identifying information is removed to protect patient privacy. Patient and provider data from the database servers 140 and 144 may be used for input factors for determining caregiver retention.

Although the database server 140 is illustrated in FIG. 1 as being an entity separate from the application server 130 the database server 140 may alternatively be a hardware component that is part of another server such as server 130, such that the database server 140 is implemented as one or more persistent storage devices, with the software application layer for interfacing with the stored data in the database is a part of that other server 130.

The database servers 140 and 144 store data according to defined database schemas. Typically, data storage schemas across different data sources vary significantly even when storing the same type of data including cloud application event logs and log metrics, due to implementation differences in the underlying database structure. The database servers 140 and 144 may also store different types of data such as structured data, unstructured data, or semi-structured data. Data in the database servers 140 and 144 may be associated with patients, groups of patients, and/or entities. The database servers 140 and 144 can comprise various schemas including those that support structured and unstructured data via SQL/Relational DBs and NoSQL technologies like Microsoft's Azure™ CosmosDB or Apache's Cassandra.

The network 150 represents the various wired and wireless communication pathways between the computing devices 120 and 122, the management system 142, the application server 130, and the database server 140. The network 150 uses standard Internet communications technologies and/or protocols. Thus, the network 150 can include links using technologies such as Ethernet, IEEE 802.11, integrated services digital network (ISDN), asynchronous transfer mode (ATM), etc. Similarly, the networking protocols used on the network 150 can include the transmission control protocol/Internet protocol (TCP/IP), the hypertext transport protocol (HTTP), the simple mail transfer protocol (SMTP), the file transfer protocol (FTP), etc. The data exchanged over the network 150 can be represented using technologies and/or formats including the hypertext markup language (HTML), the extensible markup language (XML), etc. In addition, all or some links can be encrypted using conventional encryption technologies such as the secure sockets layer (SSL), TLS (Transport Layer Security), Secure HTTP (HTTPS) and/or virtual private networks (VPNs). In another embodiment, the entities can use custom and/or dedicated data communications technologies instead of, or in addition to, the ones described above.

FIG. 2 is a high-level block diagram illustrating physical components of an example computer 200 that may be used as part of the computing devices 120 and 122, the management system 142, application server 130, and/or database server 140 from FIG. 1, according to one embodiment. Illustrated is a chipset 210 coupled to at least one processor 205. Coupled to the chipset 210 is volatile memory 215, a network adapter 220, an input/output (I/O) device(s) 225, a storage device 230 representing a non-volatile memory, and a display 235. In one embodiment, the functionality of the chipset 210 is provided by a memory controller 211 and an I/O controller 212. In another embodiment, the memory 215 is coupled directly to the processor 205 instead of the chipset 210. In some embodiments, memory 215 includes high-speed random access memory (RAM), such as DRAM, SRAM, DDR RAM or other random access solid state memory devices.

The storage device 230 is any non-transitory computer-readable storage medium, such as a hard drive, compact disk read-only memory (CD-ROM), DVD, or a solid-state memory device. The memory 215 holds instructions and data used by the processor 205. The I/O device 225 may be a touch input surface (capacitive or otherwise), a mouse, track ball, or other type of pointing device, a keyboard, or another form of input device. The display 235 displays images and other information from the computer 200. The network adapter 220 couples the computer 200 to the network 150.

As is known in the art, a computer 200 can have different and/or other components than those shown in FIG. 2. In addition, the computer 200 can lack certain illustrated components. In one embodiment, a computer 200 acting as the database server 140 may lack a dedicated I/O device 225, and/or display 218. Moreover, the storage device 230 can be local and/or remote from the computer 200 (such as embodied within a storage area network (SAN)), and, in one embodiment, the storage device 230 is not a CD-ROM device or a DVD device.

Generally, the exact physical components used in a computing device will vary in size, power requirements, and performance from those used in the application server 130 and the database server 140. For example, the computing devices 120 and 122, which will often be home computers, tablet computers, laptop computers, or smart phones, will include relatively small storage capacities and processing power, but will include input devices and displays. These components are suitable for user input of data and receipt, display, and interaction with notifications provided by the application server 130. In contrast, the application server 130 may include many physically separate, locally networked computers each having a significant amount of processing power for carrying out machine learning modeling work described elsewhere. In one embodiment, the processing power of the application server 130 provided by a service such as Microsoft Azure™ Web Services or Amazon Web Services™. Also, in contrast, the database server 140 may include many, physically separate computers each having a significant amount of persistent storage capacity for storing the data associated with the application server.

As is known in the art, the computer 200 is adapted to execute computer program modules for providing functionality described herein. A module can be implemented in hardware, firmware, and/or software. In one embodiment, program modules are stored on the storage device 230, loaded into the memory 215, and executed by the processor 205.

In this example, the machine learning module 132 in FIG. 1 provides predictions based on input data for retention of a caregiver such as the caregiver 110. The training module 134 is provided for collecting training data to determine the weights and importance of different input factors as will be explained below.

The input factors may be analyzed by the machine learning module 132 for the respective influences on retention. Data may be collected for such factors to determine their relative importance and prediction for retention as will be explained below. These factors may include: a) referral source for the hire; b) time employed at the current position; c) in service training; d) total caseloads; e) acuity levels of patients; f) type of caregiver; g) payrate/paytype; h) career growth history; i) length of travel; j) human resource metrics; k) compatibility between caregiver and patients under their care; l) availability of alternative employment m) previously worked hours by a particular caregiver and a particular patient; m) average number of hours per patient; or o) punctuality of arriving at scheduled visits. Of course, other factors may also be related to retention and be considered.

Data may be gathered for the factors described above. The input factors may be evaluated individually or in relation to each other by the example machine learning to determine the best retention outcomes. Additional factors can be derived by using a text analytics engine (e.g. from Microsoft) container that performs Named Entity Recognition (NER), relation extraction, entity negation and entity linking for English-language text. Named Entity Recognition detects words and phrases mentioned in unstructured text that can be associated with one or more semantic types, such as diagnosis, medication name, symptom/sign, or age. These may be used to create additional features/columns of data that drive more data to train the machine learning model.

Specifically, in relation to the referral source for the hire, this may include sources such as physician, agency office manager, hospital, insurance companies/payors, websites, popular job listing sites, and paper advertising. The data for the referral may be obtained from the caregiver such as the caregiver 110 or administrative systems as part of the agency management system 142 that include a human resource module that stores referral data.

Another possible input factor is the time employed at the current position. This data may be obtained from the management system 142 of the agency 116. The factor may be categorized as over 90 days or over a year or other potentially relevant time benchmarks.

Another possible input factor is the total case load for the caregiver. This factor may influence retention as caregivers generally like to see less clients for longer periods of time per visit. The desire for consistency may be captured from data relating the number of clients and length of time a caregiver is with a client. Another source of data may be payors, such as insurance companies, that may determine the period and frequency of care by limiting the number of shifts.

Another factor may be the acuity levels of the current patients seen by a caregiver. This data may be derived from either health care databases of patients relating to the caregiver such as the database 140 or database 144, or from records kept by the administrative systems relating to such patients.

Another factor may be inservice training provided by the agency 116. The training data may be kept by the administrative management system 142 of the agency 116. Inservice training allows for upskilling, learning, higher employee engagement and convenience. Thus, inservice training generates more goodwill from the employee.

Another factor may be the type of caregiver. For example, a personal care aide (lower skill) might want longer commitments with less clients while a skilled nurse (higher skill) might tolerate greater churn in their client base. This data may be collected from the administrative management system 142 or the individual caregiver through the computing device 120.

Another factor for retention may be payrate and paytype. The paytype may include a per visit scheme or a per hour scheme. Additional data may be included from external databases such as the caregiver rate compared to the median and average for their peer group in the organization or their rate compared to their peers in the industry. The payrate and paytype data may be obtained from the management system 142 for caregivers employed by the agency 116.

Another factor may be career growth history. This factor may be determined from promotion data from the administrative management system 142. Alternatively, similar data may be obtained from external databases such as professional contact websites.

Another factor is career growth history. For example, data may be obtained for how often have a caregiver received promotions in a given organization such as the agency.

Another factor may be the length of travel as longer travel may influence retention. The travel may be derived from position data from the computing device 120 associated with the caregiver 110, or from records relating to the caregiver from the management system 142. Another factor may be distance traveled per week which may be obtained from location information. The distance traveled may be determined by administrative records from the agency 116 or through position data on the computing device 120 associated with the caregiver 110. This may include monitoring late shows for clinician and monitor time and attendance in general that may be an indicator of low employee engagement.

Another input may include human resource factors such as turnover of support staff at the agency, complaints to human resources, which may be derived from data from the administrative management system 142 of the agency 116.

Another factor may be availability of alternative local sources of employment with lower skill requirements and comparable pay than the caregiver profession. Data from local sources of employment may be determined from search APIs that provide locations and data from other employers in the area.

As will be explained, many or all of the above factors may also be analyzed for prediction of retention in evaluating potential candidates for the caregiver position. These factors may include: a) referral source for the hire; b) time employed at the previous positions; c) how often job changes occurred; d) current employment status; e) payrate; f) career growth; and g) availability of alternative employment. The agency's available case loads and proximity to the candidate's location and preferences can also be used to predict the candidate's retention if hired.

This data may be reported by the candidate and or verified by administrators of the agency 116. For an example, an API may be used to access other sources such as a LinkedIn profile. The employment status may determine the individual currently unemployed or moving from another job. For example, if the candidate is consciously moving, the chances of retention are probably higher. A candidate deliberately pursuing a career growth by joining the agency has a greater chance of being retained. In this case a caregiver may decide to move to an agency based on their need to grow their career with a promotion, advancing their skills and payrate in the process. The pay rate may include data on the current rate of pay and information on whether the candidate sees a salary growth in coming with the new position. The career growth may be determined from information collected to determine whether the candidate perceives a promotion in taking the position.

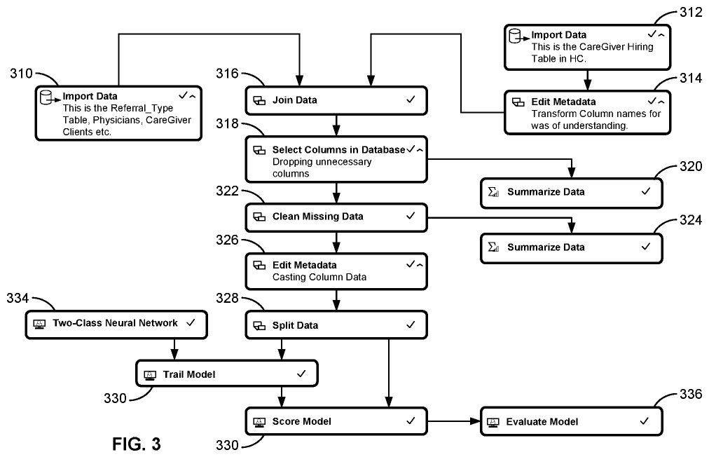

A machine learning model 132 operated by the server 130 may be trained for retention prediction both in the case of a caregiver and a potential hire for a caregiver position in this example. FIG. 3 shows a two-class neural network illustrating the process for training an example model that identifies and weighs one specific input factor for predictions of retention of caregivers. Several machine learning models may be appropriate for retention prediction including the example neural network. In practice, a data scientist may train several models (e.g., Neural Networks, Logistic Regression, Linear Regression) and decide on one after examining a variety of measures including accuracy, precision, recall or a harmonic mean of multiple factors. In this example, the data scientist takes a set of input data and splits it into a training set and a test set (also called a hold-out set). The training set might be 80% of the data and the test set might be 20% of the test set. Furthermore, within the training set the data is split 70%-30% (as an example) into two groups: a training group and validation-on-training group. A data scientist uses known techniques to sample the data in cases where there is a high amount of skew. The data scientist uses the results on the Test/Hold-out-set as a predictor of the model's potential performance under real world conditions.

In this example, the machine learning training was performed using the Microsoft Azure™ ML Studio application. In the example process shown in FIG. 3, two input features are evaluated to train a machine learning model to predict caregiver retention. The appropriate data from databases that represents the features of interest are imported for the training of the machine learning module. Thus, in this example, a first data set (310) reflecting the referral source is imported. A second data set (312) reflecting a caregiver hiring table that shows the length of employment of caregivers associated with each referral source data is also imported. In the case of the table, the data is edited in relation for metadata (314) by transforming column names for ease of understanding. Other attributes are edited to be easier to read, debug and understand. The data from the data sets is joined to cross-correlate related information (316). The columns are selected in the joined data set (318) by dropping unnecessary columns, leaving only columns with data that are interest to the model from the dataset. To perform this step a data scientist uses known techniques to correlate the features against each other and the desired output. The columns that appear to have redundant (or dependent) information are eliminated. The data is then summarized (320). The missing data is cleaned out of the data set (322). The data is the summarized again (324). This summarization is done after removing rows or data-elements or interpolating data-elements (e.g. using a mean or median to represent a missing element). In contrast, the first summarization (320) is done after dropping any columns that are unnecessary. The metadata is edited by casting the column data (326). The casting operation allows a change in the type of the data such as changing a text/string format to a numeric format.

In this example, a data lookback period is selected to suit the model objective. In this example, the desired output is a prediction of monthly retention, and therefore the data lookback period is the snapshot of the data up to a month before is used. Thus, the data during this period will be selected for training. In this example, the model is run on the last day of the month after close of business. However, the model itself can be used as necessary without waiting for end of the period. The prediction period (daily, weekly, monthly, or annually, etc.) may be fixed or may be configured.

The data relating to referral sources and the length of employment is then split (328) between a training model (330) and a Validation-on-Training score model (332). A two class neural network 334 in this example is trained. The model is trained on a portion of that data (e.g., 70%) with the known results from the current month. The resulting model is evaluated against the retention outcome data for the remaining 30% of data (336). Thus, the performance of the model is checked against the remaining 30% actuals from the current month. Once a greater than a targeted performance (e.g. accuracy >95%) is achieved from the model, the model may be used to predict caregiver retention for the next period. As explained earlier, a data scientist would actually have a hold-out test set that is removed entirely from this training/validation set. 20% of the data is removed and keep it aside as a hold out test set for testing on the model with data that the model has not seen before. Then, the remaining 80% of the data (after the hold out 20% is set aside) is further split 70%-30% for example into a training set and a validation set. The validation set, which is 30% of the 80% that is set aside for training and validation is used to test the model that has been trained with the 70% of the 80% that is used for training and validation. For simplicity here the data is only split into two groups: training and scoring (validation).

The example model in FIG. 3 resulted in no false positives and two false negatives. The example model returned high levels of accuracy and recall for retention prediction.

The process of training a machine learning module in FIG. 3 is an example with a retention case and an attrition case. Thus, the model may be trained for output of a retention prediction for the case where the employee has still been retained by the organization. The model may also be trained for output of the attrition case where an employee just left since the last window of measurement (e.g. 30 days). The model may be updated regularly as time advances based on changes in conditions. If a caregiver leaves the agency, the model is updated with this new data. If a caregiver stays, the model is updated regularly with this data. In this example, the model is trained with multiple groups of data measured at a specified point in time. The retention times may be classified as low tenured employees (less than a year); medium tenured employee (1 to multiple years)—multiple could be 4 as an example; and long tenured employees. The above techniques are used to provide the diversity of use cases to provide more robust training data. The definitions of low, medium and high tenure periods may be changed as necessary or adapted to individual agencies if necessary.

In this example, the training software operates as a supervised learning model, with inputs being the referral source data and amount of time employed by a caregiver. Based on these two inputs, the trained model attempts to classify potential and current employees as either ‘highly retainable’ or ‘not highly retainable.’ The model can also be trained to predict “attrition” or “not attrition”. Alternatively, the model can be trained to predict “retainable” versus “not retainable”

Alternatively, the model may be programmed to provide a retention score. In this example, the learning machine model may be designed as a typical regression problem. Based on the retention output data and the input data, when given data on employment history and referral source, the model predicts the probability of the caregiver being a highly retainable employee.

A model may also be run as an unsupervised learning model. The unsupervised model may be used to determine unknown factors that may affect retention of caregivers. Discovered factors that have a degree of confidence from an unsupervised model may then be used for training a supervised model for more accurate determination of retention.

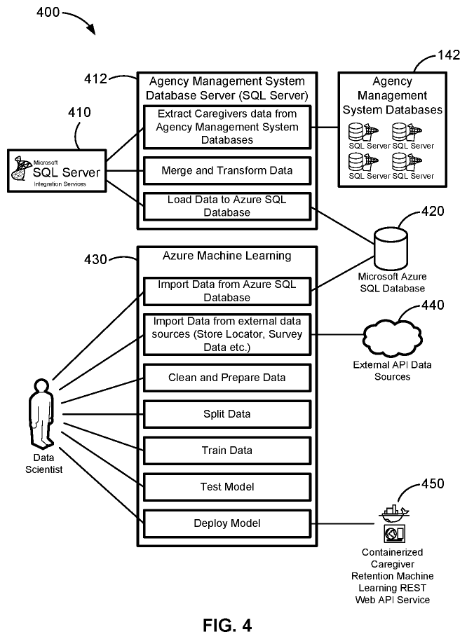

FIG. 4 is a block diagram of a general example 400 of programming a machine learning model based on multiple factors for producing a trained model for generating retention scores of care givers. Similar to the example in FIG. 3, training an example caregiver retention machine learning model is the process of using historical caregiver data to train a machine learning model that can predict caregiver retention based on at least one input factor. The process of collecting the data and training the model includes the following phases: a) loading the caregiver data from the agency management and EHR system; b) cleaning, preparing, training and testing the model; and c) deploying the model such as for a web service.

The databases and hardware include an example SQL server 410 that runs the agency management EHR system database server 412. Other types of databases may be used. The integration services server 410 may access other servers of agency management system databases 412. The SQL server 410 extracts caregiver data from the agency management system databases 412. The SQL server 410 merges and transforms the data into recognizable formats. The SQL integrations server 410 then sends the data to a database 420. In this example, the database 420 is a Microsoft Azure™ SQL database. A machine learning module 430 selects and trains a machine learning model.

In this example the machine learning model is a neural network. The neural network may be a multilayer perceptron (MLP) neural network model with the use of one or more hidden layers. The neural network MLP model adjusts internally derived calculated weights between each of the established node connections by minimizing an error function against actual values during the training process. Other examples of machine learning models may include a decision tree ensemble, a support vector machine, a Bayesian network, logistic regression, linear regression, or a gradient boosting machine. Such structures can be configured to implement either linear or non-linear predictive models.

The loading of caregiver and related data relevant for training the model is executed on the agency management EHR database servers 412. During this phase historical caregiver data is extracted from the agency management system 142. The data is transformed and loaded in a data store accessible to the machine learning environment. A SQL Server Integration Services (SSIS) package is used for extract/transfer/load (ETL) processing. This can be expanded in the future to cover other database and ETL technologies or (extract load transform (ELT) or live real-time streaming data. In the example above, the Azure™ SQL database 420 is utilized for intermediate data storage, but other data store technologies can be used if needed.

During the loading phase the SSIS package extracts caregiver related data from the agency management system databases 412. The extracted data is comprised of the features (inputs) of the machine learning model and the target (output) of the model. In this example, the target output is retention data of the caregivers that are associated with the features.

For the model to be accurate, only data that is available prior to the prediction should be used. To accomplish this, active caregiver data is collected at different snapshots of time. A lookback period is established that determines the period prior to the snapshot point that will be used to extract caregiver related data. The lookback period is typically one month prior to the snapshot point but the duration of the lookback period can be adjusted depending on the overall requirements.

Examples of data extracted from the lookback period that are potential factors may include the home GPS location of the caregiver; the hiring referral source of the caregiver, the average daily pay amount during the lookback period, the average number of clients visited by the caregiver per day during the lookback period; the average number of hours worked per day during the lookback period; the average number of visits per day during the lookback period; the average distance travelled per day during the lookback period; the acuity of the patients that the caregiver has been assigned, the number of days the caregiver was employed up until the snapshot point. In addition to the features above, the number of days that the caregiver was retained after the snapshot point is also extracted during this step. This is the information that the model will learn to predict (target). Fewer input features may be selected for simpler models. Additional input factors may also be added for more complex and accurate predictions.

The relevant data extracted from all the agency management databases 142 is merged and transformed into a standard format and stored into a single database 420. A structured SQL database format may be used initially to store the data, but other file formats can be used later to optimize the process. The transformation includes all steps needed to clean, prepare train and test the model.

In this example, Azure™ Machine Learning Studio is used as the machine learning module 430. The machine learning module 430 is used to train the machine learning model with the extracted and transformed data but other similar technologies can be utilized. The following steps are used in this phase. The transformed data is extracted from the Azure™ SQL Database 420. The data extracted from the Agency Management Databases supervised by the management system 142 and stored in the Azure™ SQL Database 420 is imported into the Azure™ Machine Learning environment 430 for processing.

Data is also imported from external sources 440 and loaded into the machine learning module 430. Examples of external data include store locator data and survey data for purposes of determining a comparable employment input factor. Thus, store locator data may be imported from mapping solutions like Google Maps and include the number of stores or businesses open in a specific location. The data is used to calculate the number of competitive job opportunities close to the caregiver's home GPS location. This data may be used as a feature (input) for the model. Survey data may be imported from third party survey companies that survey caregivers and clients. The data will be used as a feature (input) for the model. Other external data from health care databases such as the database 140 in FIG. 1 for additional patient data, or additional databases storing data relating to the caregiver 110, can be imported if required.

The machine learning module 430 cleans and prepares the data for training the model. Some examples of cleaning the data include: identifying data that is not formatted properly and removing such data; identifying rows with missing data and remove or update missing data with default values; identifying columns (features) that contain single or very few values and remove such values; identifying and removing duplicate data; and identifying and removing features that have very low correlation to the target.

In this example, the data is split into two datasets by a data scientist for the machine learning module 430. The first dataset is used to train the model and the second dataset is used to test the model (test/hold out set). A common practice is to split the data so that 70% of the data will be used to train the model and 30% of the data will be used to test the model. Other split percentages can be used if needed. The 70% data is further split into training data and validation data. The training data is used to train the ML model and the validation data is used to test against the training. The test/hold-out 30% data is used for final testing of the model to simulate close to real world situations. The data scientist uses the results on the test/hold outset as a predictor of the potential performance of the machine learning under real world conditions.

A data analyst initiates the training of the model based on the first dataset. Training the model includes choosing an algorithm model type, assigning the training data set, and training the model. Different classification models, regression models and clustering models may be evaluated and the model with the best accuracy will be used. In this example, a classification model is used initially due to its simplicity with a goal of utilizing different models later as the product matures. Once the model is selected, the training dataset is assigned to the model and configure the features (inputs) and target (output). The model is then trained based on the configured algorithm

The model generated in the training process is evaluated based on the generated test dataset. During the evaluation, evaluation scores are generated that help data scientists to measure the accuracy of the model. The evaluation scores for different models are compared to select the best algorithm. The training and evaluation are repeated with different algorithms and features to identify the options that produce the most accurate model.

Thus, the machine learning module 430 obtains the merged and transformed data from the database 420. The machine learning module 430 imports data from the SQL database. The module 430 may also import data from external sources such as the patient database 140 in FIG. 1. The external data may constitute additional input factors for the machine learning. The machine learning module 430 cleans and prepares the data. In this example, the machine learning module 430 splits the data so some data is used to train/validate the model while other data is used as a hold-out to test the model. The module 430 performs testing of the trained model.

In this example, the model may be deployed via a network to computing devices with web access such as a workstation. Once a model is selected, in this example the model is deployed as a web service 450. A REST web service is generated that takes values for each feature as inputs and outputs the prediction value (retention score) from the trained model. The API is deployed as a containerized web service, but other options may be added. In one such rendering the machine learning model is embedded directly with the rest of the application that manages the home health care agency 116. This application could be an Electronic Health Care Records (EHR) system.

The system in FIG. 4 may be used to evaluate retention based on various input factors that are used to train the machine learning model. For example, a model using the system in FIG. 4 may be construed based on the six specific factors of patient acuity, compensation, caseload, the payor, the referral source, and the past tenure. When a retention score indicates a caregiver is likely to leave their employment, a root cause of one or more of the factors may be determined by the machine learning model. A primary factor may be determined from the multiple input factors that contribute to the change in retention condition. When the machine learning model is deployed, additional inputs and resulting retention data may be retained for fine tuning the learning process and improving accuracy of the model during its use by the agency management system. Such retraining may be performed periodically after sufficient data is gathered.

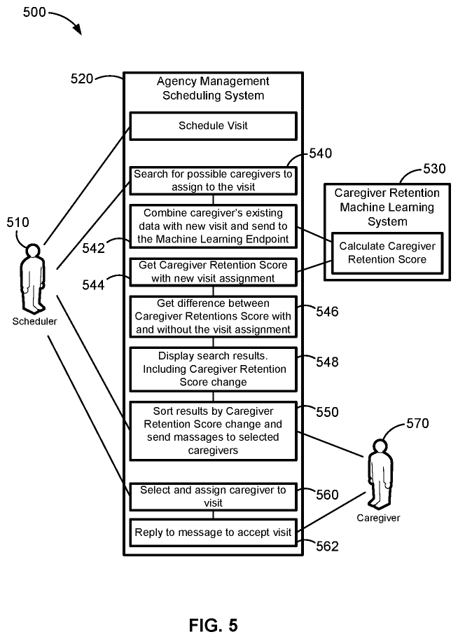

The results of the model may be developed to provide useful information to the agency 116 or other employer for the caregiver 110. In one application, a manager or administrator might go to a dashboard regularly (daily, weekly, monthly) to view the status of retention which is represented visually. In another case, the retention machine learning model can be used in a real-time application. FIG. 5 shows an example system 500 that allows an administrator such as a scheduler 510 to use a determined caregiver retention score for scheduling caregivers employed by an agency. The caregiver retention score generated using the disclosed processes can be used as a search attribute when searching and assigning caregivers to visits. The system 500 includes an agency management scheduling system 520 that may be a component of the management system 142 in FIG. 1. The system 500 also includes a machine learning system 530 for predicting caregiver retention in the form of an output caregiver retention score. The machine learning system 530 may be trained with data collected as explained above. The manager can also regularly view a dashboard generated by the example application that provides a view at the corporate level, regional level, agency level and caregiver level to view changes from the previous time period to retention and also the primary driving factor or all the factors driving the change in retention if necessary.

The scheduling system 520 first searches for possible caregivers to assign to the visit (540). The scheduling system 520 then adds the caregiver retention score from the machine learning system 530 is added to the existing caregiver search attributes that schedulers use to match caregivers to visits (542). Some other examples of attributes typically used to search for caregivers are caregiver availability, compatibility match between the client and the caregiver, travel distance etc. Adding the caregiver retention score to the caregiver search attributes allows schedulers to review how their scheduling decisions can affect retention of a caregiver before assigning a caregiver to a visit. Thus, the scheduling system 520 obtains a caregiver retention score from the machine learning system 530 with data relating to a new visit arrangement (544). The scheduling system 520 then obtains the difference between the caregiver retention score with and without the visit assignment (546). The scheduling system 520 displays the search results that include the change in retention score based on the visit (548). The scheduling system 520 the sorts results by caregiver retention score changes based on the visit (550).

In this example, the scheduler 510 may select a caregiver with minimal or no change in caregiver retention score or an improvement in the score from making the assignment. Alternatively, the scheduler 510 may consider other factors in selecting a caregiver. Alternatively, the scheduling may be automated based on a rule or policy based application. The scheduling system 520 includes a caregiver selection interface 560. The selection of the caregiver from the interface 560 is sent to a communication module 562 that contacts a selected caregiver 570 via a text or email or other media.

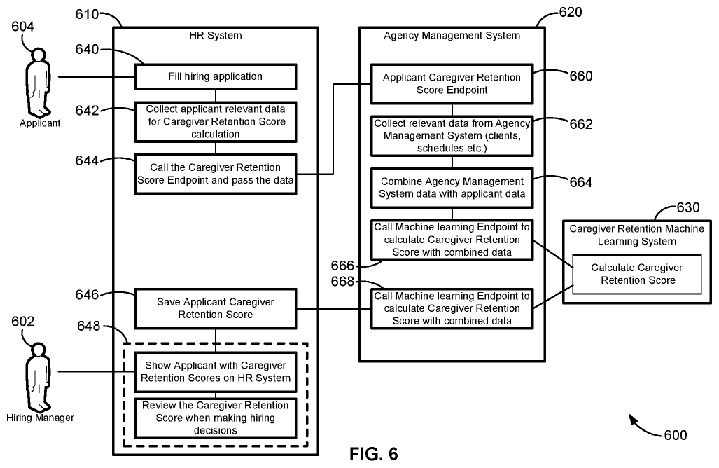

Another potential application is using retention scores obtained by the machine learning module for hiring. FIG. 6 shows an example human resources evaluation system 600 that may be used by a hiring manager 602 to evaluate a candidate 604. The human resources evaluation system includes a human resources system 610, an agency management system 620, and a machine learning module 630.

The human resources system 610 includes an application interface 640 that collects hiring application data in the form of inputs by the candidate 604 to an interface or by other means. The human resources system 610 includes a collection module 642 that collects the relevant data from the interface 640. A data interface 644 is in communication with the agency management system 620. The human resources system 610 also includes a data output interface 646 for displaying the retention prediction for each candidate, and a review tool 648 to assist hiring managers to make hiring decisions.

Relevant data from the collection module 642 is read by a caregiver retention score endpoint 660. A collection module 662 collects relevant data from the agency management system 600 such as clients, schedules and other input data relevant to the retention score. A combination module 664 combines agency management system data with the applicant data. The combined data is passed to a machine learning interface 666. The machine learning interface 666 passes the relevant data as inputs to the machine learning system 630. The input data is analyzed by the machine learning system 630 to produce a caregiver retention score for the candidate 604. The produced retention score is returned by a score interface 668.

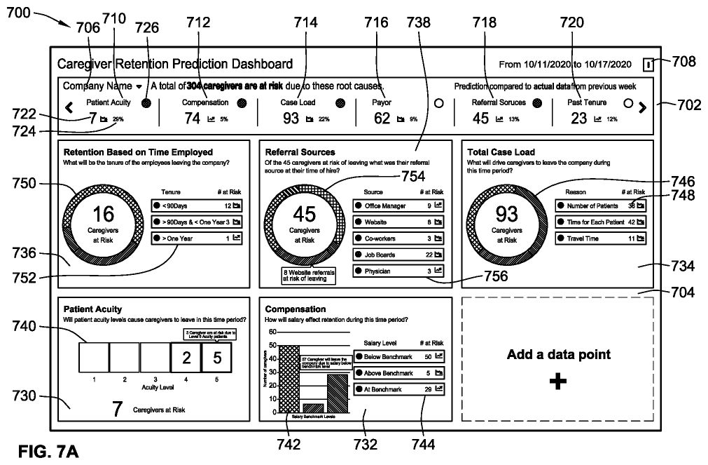

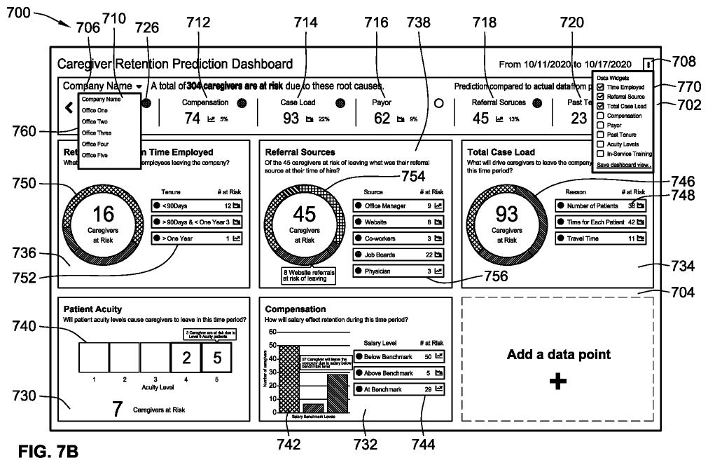

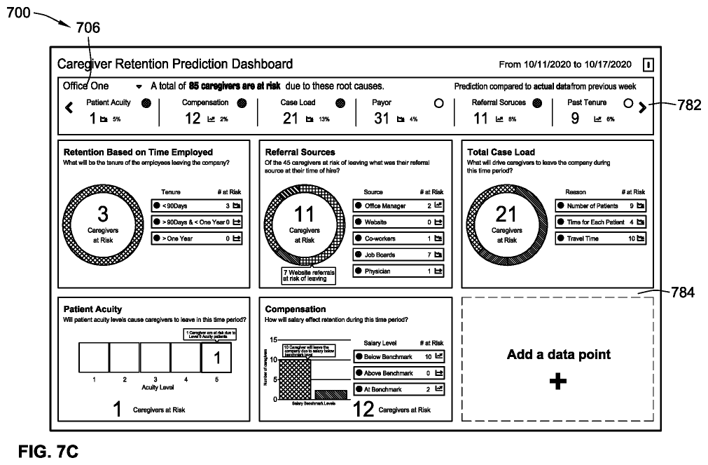

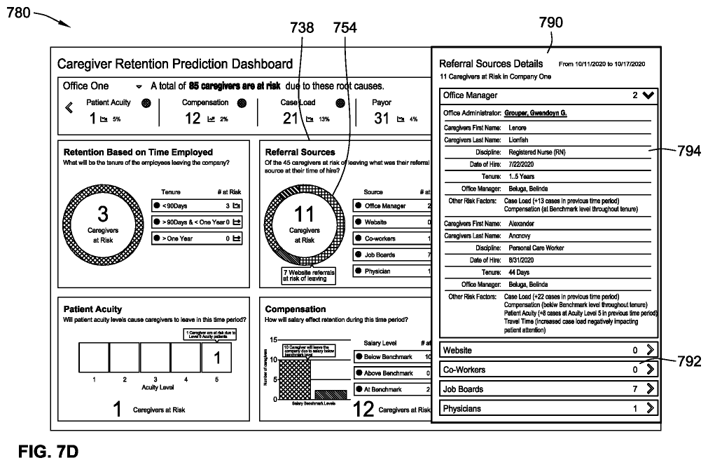

The score is received by the data output interface 646. The retention score is used by the review tool 648 to generate a display. The review tool 648 presents the retention score along with other information relating to the candidate 604 to the hiring manager 602 as will be explained below in relation to FIGS. 7A-7D. For example, the score may be presented as part of a dashboard interface that provides a high-level corporate or agency wide view which can be drilled down to the individual caregiver level.

The caregiver retention score may thus be used by the human resources system 610 to allow agencies to proactively select the best caregiver candidates based on projected retention scores of candidates. To calculate the projected caregiver retention score, the candidate data collected during the hiring process are combined with the data of the agent. The generated caregiver retention score from the machine learning system 630 are targeted and tailored to the compatibility between the applicant and the agency. For example, the caregiver retention score could be negatively affected if the caregiver's home address is located too far away from the clients of the agency.

The determined retention scores may have other uses. For example, once the model determines if a caregiver is going to leave, the model can provide cost analysis on new hire. The cost analysis may include analysis of the costs of onboarding, cost of training, severance, finding a new candidate etc.

Alternatively, upon receiving the output, the care retention scores may also suggest changes to various factors that may result in a caregiver becoming highly retainable, for example, increasing the hourly wage.

Upon receiving output of different caregivers at different facilities, the model may determine if certain features considered ‘cultural’ features might increase or decrease retention. This information may allow an agency to replicate facilities that tend to have higher retention. The information may also allow the agency to configure client-caregiver match process. The match may be made by an employee or a specialized application that automates recommended matches. The client/caregiver compatibility match may be based on cultural norms and personality differences from data relating to the patient and caregiver.

Upon receiving the output, the software may create and update a visualization of caregiver job satisfaction or caregiver departure threat. This visualization could be updated in real-time by receiving data from, for example, mobile phones that include tracking information, including a scheduling application that lists addresses of clients. The software may be able to pull that address information and process travel times or distance between client locations. If increasing commute times or time spent in traffic is increasing the threat level, the software may shift the schedule to reduce the caregiver workload.

As explained above, the determined retention score for a caregiver or a potential caregiver may be used for different agency management functions. FIG. 7A is a screen image of an example dashboard interface 700 for management of an agency. The interface 700 includes a root cause banner field 702, and an analysis area 704 allowing display of data point windows. In this example, the root cause banner field includes a summary of the total number of caregivers at risk for the agency. The total number of caregivers at risk is determined by the retention scores for those caregivers output by the machine learning model 132 that exceed a certain threshold value. The interface 700 also includes a company pull down menu 706 that allows a breakdown by individual offices of the agency. The interface also includes a data widgets selection 708 that allows the selection of different data filters.

In this example, the banner field includes tiles 710, 712, 714, 716, 718, and 720 that each display the number of caregivers of the total number of caregivers at risk due to a specific factor or root cause. Thus, in this example, the tile 710 displays a number of caregivers 722, a trend icon 724, and a data analysis selection 726, for the root cause of patient acuity. As explained above, the retention score of a caregiver may be associated with one of the input factors as the root cause. Standard available libraries (embedded into systems like Microsoft Azure™ ML Studio and Python language ML libraries) provide the ability to find the root event that drives a change in condition. This is input to the manager who can use this along with other factors (e.g., change in managers at the office, change in financial conditions) to determine the true root cause. The number of caregivers 722 is the number of caregivers falling under the root cause. The trend icon 724 indicates whether the number of caregivers for the root cause is increasing or decreasing over a period of time such as a week. For example, in FIG. 7A, 7 caregivers are at a risk of leaving due to patient acuity increases and this represents a 29% increase (worsening condition is a downward trend and the color red represents this) from the last time period.

Similar to the tile 710, the tile 712 shows the number of caregivers and the trend due to the root cause of compensation. The tile 714 shows the number of caregivers and the trend due to the root cause of case load. The tile 716 shows the number of caregivers and the trend due to the root cause of payors. The tile 718 shows the number of caregivers and the trend due to the root cause of referral sources. The tile 720 shows the number of caregivers and the trend due to the root cause of past tenure. Additional tiles may be displayed through arrows at either end of the banner field 702.

Selecting a data point analysis selection such as the selection 726 will display an analysis window in the analysis area 704. In this example, the selections of the tiles 710, 712, 714, and 718 have been selected. Thus, corresponding analysis windows 730, 732, 734, and 738 are displayed in the analysis area 704. An additional analysis window 736 is displayed in the area 704.

Each of the analysis windows 730, 732, 734, 736, and 738 show more detailed information for caregivers falling under a specific root cause. For example, the window 730 shows a graph 740 of the number of caregivers that are treating patients at the different patient acuity levels. The window 732 shows a bar graph 742 that breaks down the caregivers falling under the compensation root cause into above, below and at a salary benchmark level. The window 732 also includes a table 744 that lists the number of caregivers at risk who are below, above and at benchmark salaries levels, as well as the trend from the previous time period. The window 734 relating to total case load includes a graph 746 representing different metrics of total case load and the number of caregivers related to each metric. While a caregiver may be at risk due to multiple causes, the caregivers are categorized at this level to the most-likely or primary risk category associated with the caregiver. The manager can drill down into the details for a particular caregiver and determine the multiple risk factors that are at play representing the change in condition. The window 734 also shows a table 748 that lists each metric of case load and the corresponding number of caregivers who are risk relating to each metric as the trend from the previous time period.

The window 736 relates to the root cause of retention based on time employed. The window 736 includes a graph 750 representing different periods of tenure and the number of caregivers in each period of tenure. The window 736 also shows a table 752 that lists each period of tenure and the corresponding number of caregivers who are risk as well as the trend from the previous time period. The window 738 relating to referral sources includes a graph 754 representing different sources of referral and the number of caregivers related to the source of referral. The window 738 also shows a table 756 that lists each source of referral and the corresponding number of caregivers who are risk relating to each source of referral as well as the trend from the previous time period.

FIG. 7B is a screen image of the example interface 700 in FIG. 7A with the selection of the company pulldown menu 706 and the data widgets selection 708. The company pulldown menu 706 when selected displays a list 760 of the different offices of the company. Selecting one of the offices will produce an interface of the caregivers specific to that office. Other filtering may be provided such as geographic region or other needs of a manager. The default will be to display company wide data. While the views here represent one type of drill down hierarchy, any level may be created depending on the customer's needs: Corporate, Regional, sub-regional, agency sub-group, individual agency, caregiver groups, individual caregivers and the like.

Selecting the data widgets selection 708 displays a data widgets menu 770. The data widgets menu 770 includes different data filters relating to the input factors/root causes. In this example, the menu 770 lists the time employed, referral source, compensation, payor, past tenure, acuity levels, and inservice training. Selecting one or more of the inputs in the menu 770 causes the view on the dashboard to be limited to these factors. As an example, an agency might only want to focus in on acuity levels and examine the details of this one factor. The user can elect, through the dropdown menu 770, to display only one visualization in the dashboard, or the user can select any combination of visualizations. The dashboard will grow (or shrink) as required to fit the selected cards.

FIG. 7C is a screen image of an example management interface 780 for a specific office of an example agency selected form the company pulldown menu 706 in FIG. 7B. The interface 780 displays the same data as the interface 700 for the caregivers specific to the selected specific office. Thus, the interface 780 includes a banner field 782 breaking down the number of at risk caregivers in the specific office by root causes and a details area 784 that displays tiles for selected root causes from the banner field 782.

FIG. 7D is screen image of an example referral sources pop-up window 790 accessed from the example management interface 780 in FIG. 7C. In this example, the referral source tile in the details area is selected by either clicking on the referral source tile or hovering over the tile to display the pop-up window 790. In this example, the user has specifically clicked on the office manager segment of the graph 754 and thus, the pop-up window 790 shows caregivers under office manager referral sources. The pop-up window 790 includes relevant data for each of the caregivers under each of the referral sources listed in the tile. Thus, the pop-up window 790 includes tabs 792 that each correspond with the listed referral sources including an office manager, website, co-workers, job boards or physicians. Each of the tabs 792 also includes the number of caregivers that were referred from that source. When one of the tabs is expanded such as the office manager tab, a listing 794 of data for each caregiver that is at risk who was referred to this agency/office by an office manager is shown. The listing 794 includes data such as the name of caregiver, the date of hire, the tenure, the office manager and other risk factors derived from the collected data.

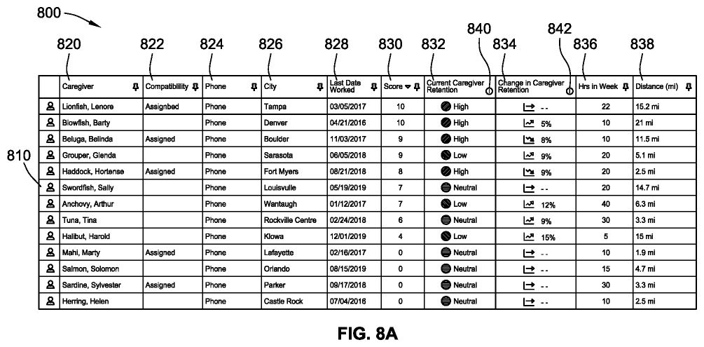

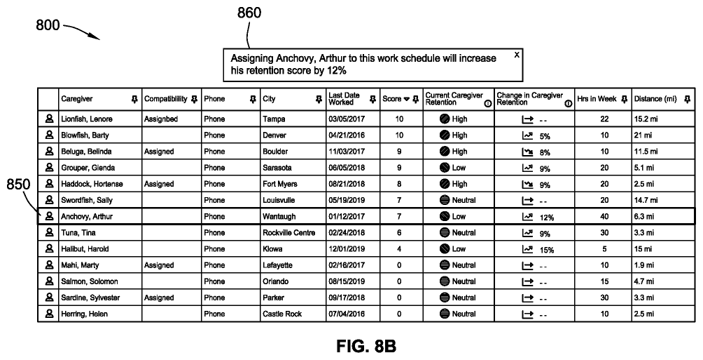

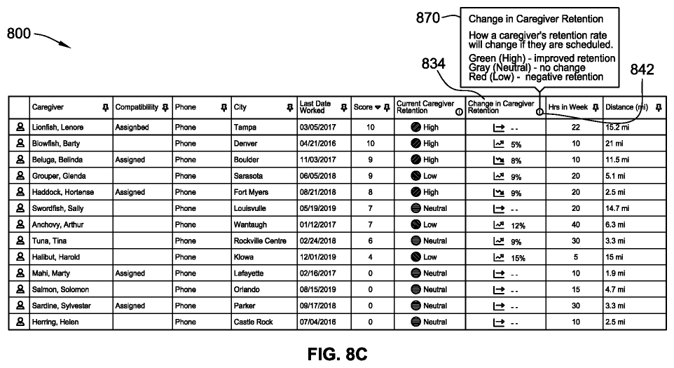

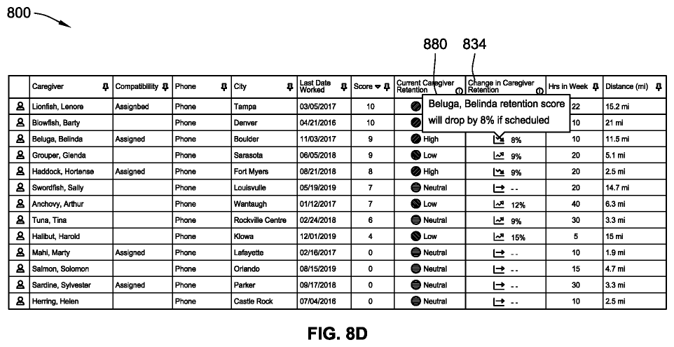

FIG. 8A is a screen shot of an example scheduler interface 800 that is based on real-time use of the machine learning algorithm during active scheduling of care. While the previous examples are more applicable to a regular dashboard-oriented interaction with the machine learning algorithm, in this case the interaction is potentially driven by active alerts and a real-time insertion of this capability into the active workflow of the administrators. In this example, the scheduler interface 800 may incorporate retention scores to schedule caregivers. The interface 800 includes a row listing of potential caregivers 810 that can be assigned to the patient's home visit. Different columns allow an administrator to view valuable information for scheduling and other administrative purposes. The columns include a name column 820, a compatibility column 822, a phone column 824, a city column 826, a last date worked column 828, a score column 830, a retention column 832, a change in retention column 834, an hours column 836, and a distance column 838. Except the columns 832, and 834, the remaining columns are populated based on a combination of data related to the specific caregiver from the caregiver database 144 in FIG. 1 and new intended assignment of a patient to a given caregiver. In this scenario an administrator at an agency is trying to match a patient home visit with a caregiver. In this case, the distance column represents the distance between the caregiver's home and the home of the patient that is being scheduled by this dashboard. The retention scores for each of the caregivers are calculated using the machine learning model 132 in FIG. 1 explained above. The retention attributes displayed in columns 832 and 834 show the resulting change in retention from making the assignment of the new incoming patient to a particular caregiver.

The score column 830 displays a compatibility score between the client and the caregiver by comparing the client attributes to the caregiver attributes. In the example below, the requested client attributes for the search are Spanish with a compatibility score of 8 and a chef with a compatibility score of 2. Each caregiver that is returned from a search will have a compatibility score assigned to depending whether they have a Spanish and chef compatibility. The numbers 8 and 2 have meaning only relative to each other. The scheduler/manager attributes these weights during the interview of the patient or the patient's family. The numbers only have meaning relative to each other.

The compatibility column 822 shows the compatibility of a caregiver and client. By default, a caregiver and client that have been scheduled together in the past will have a compatibility of Assigned. However, administrators can assign other compatibility values (example: excellent, poor) between the client and the caregiver. If no compatibility has been established between the client and the caregiver the column will be empty. The interface 800 may be generated for caregivers based on different search criteria such as by compatibility score. In this example, the list of caregivers is sorted by compatibility score. The distance column 838 shows the distance between the residence of the caregiver and the new patient location. In this example, the caregiver retention score is categorized into a high retention, neutral, or low retention range. The specific range is then displayed in the retention column 832. The designation may also be provided a color such as green for high and red for low for better visibility to the viewer. The change in caregiver retention column 834 shows the change in percentage between the current caregiver retention score and the resulting new retention score if the user decides to assign the caregiver to the new patient visit. Both columns 832 and 834 have respective information icons 840 and 842 that when clicked, provide an explanation to the user/administrator of the meaning of the data in the column.

FIG. 8B shows the interface 800 in FIG. 8A when a particular caregiver 850 is highlighted indicating a potential assignment of a specific caregiver (Arthur Anchovy) to a new patient under their care. In this example, the selection causes a retention score to be determined assuming the assignment of the caregiver. A pop-up window 860 is displayed that describes the effect of the assignment on the retention score. In this example, 860 explains that assigning Arthur Anchovy to the new patient visit will increase his retention score by 12%.

FIG. 8C shows the selection of the information icon 842 in the interface 800 in FIG. 8A. When the information icon 842 is selected, a popup window 870 is displayed that includes information on the definition of the change in caregiver retention and details on the three potential trends. In this example, the trends are improved retention, no change in retention, or negative retention. As explained above, the icons in the change in retention score column 834 may be color coded. In this example, an improved retention is shown in green, a no change in retention is shown in gray, and a negative retention is shown in red.

FIG. 8D shows the interface 800 in FIG. 8A, when a specific indicator of retention change is selected for a particular caregiver. In this example, the user hovers the mouse cursor over an icon in the change in retention column 834. A window 880 is displayed that shows the effects on retention if a particular assignment is scheduled for that caregiver. If the administrator decides to assign the particular caregiver (Belinda Beluga) to the patient visit, they will click on the row listing that caregiver. Once clicked, the selected caregiver will be assigned to the patient visit and they will be taken back to a visit details page where they will be able to save the scheduling assignment

Thus, the above described process provides a model that can determine, in real-time, the likelihood that any given caregiver will depart by using the previously described trained machine-learning model which is trained using several factors that may influence caregiver retention. A management interface adapts in real-time to measured information associated with a particular caregiver and factors of a particular caregiver. As described earlier the training and retraining is done at regular intervals (e.g., daily or monthly) or is also done every time there is a change in one of the attributes that affect the model. As described earlier, the model uses a deep understanding of the attributes that affect caregiver retention in home-centric healthcare environments. It uses these uniquely identified insights (attributes) for training a machine learning model to predict the retention of an individual caregiver.

Another potential input feature or features may be derived from written notes created by the caregiver, such as progress notes that track clinical progress. Also, notes about retention from a manager, or any other types of unstructured notes that can be converted to structured features to provide additional data for training the model. Some of the data may feed existing features. For example, notes that describe a change in acuity for a patient by the caregiver can be used to bias the score to including the implications of this narrative. The notes may result in the creation of new features such as a metric that describes the tone and level of unhappiness with this particular assignment of patient to this caregiver. As an example, the patient might not be cooperative and follow the caregiver instructions. This can lead to frustration on the part of the caregiver and might be captured in a text notes within the application. This note might also be the result of a voice based interface where the caregiver simply speaks into the client device 120 shown in FIG. 1. Also, there might be notes that the agency keeps about the caregiver engagement. These might be more of a narrative of complaints lodged by the caregiver about their assignments or workplace treatment that could be critical in understanding caregiver engagement. These notes created by the caregiver (patient centric notes) and by managers of the caregiver (caregiver engagement centric notes) can also be used to created structured information to train our machine learning model and enhance our predictive outcomes.

Notes may be used to derive diagnosis codes or other attributes by use of a text analytics standard engine that uses data from unstructured notes to derive additional data columns that drive caregiver retention. Recorded notes may be stored and then a standard voice-to-text convertor may be applied. The text output of the voice-to-text converter is then converted to text analytics.

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

What is claimed is:

1. A computer-implemented method for training a machine learning model to predict caregiver retention, the method comprising:
receiving a set of data from at least one database storing caregiver data;
normalizing the set of caregiver data to create a modified set of caregiver data, wherein the modified set of caregiver data defines at least one parameter for each of the plurality of caregivers from the set of caregiver data and a corresponding employment status for each of the plurality of caregivers;
generating, based on the correlation and the modified set of caregiver data, a training set of caregiver data that includes at least one parameter that correlates with employment status;
splitting the training data into a first set of data to train and validate a machine learning model and a second set of data to test the trained machine learning model;
training the machine learning model using the first set of data, wherein the training comprises predicting an employment status associated with the at least one parameter; and
evaluating the accuracy of the trained machine learning model with the second set of data.

2. The computer-implemented method of claim 1, further comprising determining a lookback period for the set of caregiver data, wherein the lookback period is based on employment status over a preceding predetermined period of time, wherein the employment status includes an indication of retention or an indication of departure after the lookback period.

3. The computer-implemented method of claim 1, further comprising transforming the set of caregiver data into a structured database format optimized for input into the machine learning model.

4. The computer-implemented method of claim 1, wherein the at least one parameter includes at least one of a) referral source for the hire; b) time employed at the current position; c) in service training; d) total caseloads; e) acuity levels of patients; f) type of caregiver; g) payrate/paytype; h) career growth history; i) length of travel; j) human resource metrics; k) compatibility between caregiver and patients under their care; l) availability of alternative employment; m) previously worked hours by a particular caregiver and a particular patient; n) average number of hours per patient; or o) punctuality of arriving at scheduled visits.

5. The computer-implemented method of claim 1, further comprising evaluating a potential caregiver candidate based on the prediction of the machine learning model of future employment status, wherein the machine learning model is provided an input of the at least one parameter for the potential caregiver candidate.

6. The computer-implemented method of claim 1, further comprising evaluating a caregiver for an assignment to a patient based on the prediction of the model of future employment status, based on an input of the at least one parameter for the caregiver.

7. The computer-implemented method of claim 1, further comprising determining a compatibility score representing a matching criteria between the caregiver and the patient.

8. The computer-implemented method of claim 1, wherein the future employment status is expressed in a numerical retention score.

9. A method of generating a retention score for a caregiver, the method comprising:
training a machine learning model using a first input factor to predict retention, the training including providing a dataset of caregivers having the first input factor and an associated employment status;
evaluating the machine learning model to determine the associated employment status generated by the model meets a predetermined accuracy level;
inputting an input factor relating to the caregiver to the trained machine learning model to predict retention; and
determining a retention score of the caregiver based on the predicted retention.

10. The method of claim 9, wherein training the machine learning model includes using a plurality of input factors including the first input factor to predict retention, the method further comprising determining a root cause corresponding to one of the plurality of input factors responsible for the retention score.

11. The method of claim 9, further comprising:
inputting an input factor value of the input factor relating to each of a plurality of caregivers;
determining a retention score of each of the plurality of caregivers; and
determining a number of the plurality of caregivers having a retention score under a predetermined threshold value indicating a likelihood that the number of caregivers will leave an agency.

12. The method of claim 9, further comprising:
comparing the determined retention score to a predetermined threshold; and
providing a warning if the retention score is under the predetermined threshold indicating the caregiver is at risk to leave an agency.

13. The method of claim 9, further comprising generating data from unstructured notes via a text analytics engine generating data, wherein the first input factor includes data generated by the text analytics engine.

14. A method for determining retention scores for a plurality of caregivers, the method comprising:
collecting caregiver data from a caregiver database, the caregiver data including an input factor relating to employment status for each of the plurality of caregivers;
inputting the input factors into a machine learning model trained using the input factor to predict retention, the training including providing a dataset of caregivers having the input factor and an associated employment status;
generating a retention score of each of the plurality of caregivers from the machine learning model; and
displaying the retention scores of at least some of the plurality of caregivers on a display.

15. The method of claim 14, further comprising:
accessing the machine learning model to generate a later set of retention scores of each of the plurality of caregivers at a later time than the determination of retention scores;
determining a change in retention scores between the retention scores and later retention scores; and
outputting changes in the input factors corresponding to caregivers with a change in retention scores.

16. The method of claim 14, further comprising generating an interface on the display providing aggregated retention scores for the plurality of caregivers.

17. The method of claim 14, wherein the interface allows the ordering of the plurality of caregivers by different levels of organizations associated with the caregivers.

18. The method of claim 17, wherein the organizations include corporate divisions or, geographical regions.

19. The method of claim 14, further comprising classifying the plurality of caregivers into different retention risk categories based on the later retention score.

20. The method of claim 19, wherein the interface includes changes in the number of caregivers in different retention risk categories based on the later retention score.
